(19) United States
(12) Patent Application Publication
(10) Pub. No.: US 2015/0277464 A1
(43) Pub. Date: Oct. 1, 2015

(54) HYBRID RENEWABLE ENERGY SUPPLY SYSTEM

(71) Applicant: Feng Chia University, Taichung (TW)

(72) Inventors: Chiu-Yue Lin, Taichung (TW); Ping-Jei Lin, Taichung (TW); Chyi-How Lay, Taichung (TW); Chin-Chao Chen, Taichung (TW); Chen-Yeon Chu, Taichung (TW); Huang-Chih Lu, Taichung (TW); Chueh-Chen Wu, Taichung (TW)

(21) Appl. No.: 14/230,006

(22) Filed: Mar. 31, 2014

(51) Int. Cl. G05F 1/66

(52) U.S. Cl. CPC G05F 1/66

(57) ABSTRACT

A hybrid renewable energy supply system is mainly applied to a stationary or mobile actual mass-production power generating operation to transform a lot of organic waste into electric energy using a gaseous biomass power plant, and may be combined with renewable energy such as solar energy, wind energy, micro-hydro energy and the like, thereby providing a self-operating system without using mains. The gaseous biomass power plant can use an organic material source, such as food wastewater, livestock farming wastewater, agriculture waste or the like, to generate a biomass gas, thereby possessing dual effects of waste recovery and clean energy production. The hybrid renewable energy supply system comprises at least one renewable energy device for transforming nature energy into electric energy; and an electric energy control device, which connects the renewable energy device to a mains grid and controls storage and output of electric energy of the renewable energy device.

### BACKGROUND OF THE INVENTION

1. Field of the Invention

The invention relates to the green power technical field, and more particularly to a hybrid renewable energy supply system, in which a gaseous biomass power plant, a wind power plant, a solar power plant and a micro-hydro power plant are combined, wherein the gaseous biomass power plant has the characteristic of self-operating without mains.

2. Description of the Prior Art

The gaseous biomass energy refers to the hydrogen and methane, and is mainly the methane in the conventional anaerobic wastewater treatment process. The complicated organic substances in the wastewater need to be transformed into the lower fatty acids (e.g., acetic acid, propionic acid and butyric acid) by other bacterium so that the methanogens can be transformed into the methane. However, the efficiency thereof is not high. Recently, the use of the two-phase fermentation process is increasingly attended. The principle of the two-phase fermentation process is to transform the hydrolysis, acidification and methanation into a hydrolysis acidification phase and a methane phase. In the front end, the bacteria grow rapidly to rapidly decompose the organic substance in the wastewater and generate the hydrogen. In the rear end, the methane can increase the reaction rate by rapidly using the front-end decomposed lower fatty acid.

In a country where the energy is insufficient and the industry is progressing, the problem of a lot of organic wastewater to be processed is present. Thus, using the hydrogen microorganisms (anaerobic biometrics) and the methanogens to decompose a lot of organic wastewater can solve the long standing organic wastewater problem and generate the gaseous biomass energy, and is indeed the technology worth developing.

The electric power source for the typical wastewater processing facility is mainly based on the mains. At present, a lot of renewable energy, such as solar energy, wind energy and waterpower energy, have been commercialized. The conventional organic wastewater treatment process can be upgraded to an electric power supply device if all the renewable energy can be integrated without the need of the mains generated by the fossil energy so that the conventional organic wastewater treatment process can perform the self-operating. Alternatively, it is desired that the waste heat generated upon electric power generation is recovered to keep the temperature of the biomass gas generating device (anaerobic fermentation reactor), and that the gaseous biomass energy, which is generated upon processing of the wastewater, the electric energy, which is generated by the gaseous power plant, and the electric energy, which is generated by other renewable energy devices can be evaluated by an electric energy control device and fed back to the mains grid.

### SUMMARY OF THE INVENTION

It is therefore an object of the invention to provide a hybrid renewable energy supply system, which may be applied to a stationary or mobile actual mass-production power generating operation to transform a lot of organic waste into the electric energy, and may be combined with the renewable energy, such as solar energy, wind energy, micro-hydro energy and the like, to enhance the overall electric power generating efficiency, and solve the conventional drawback that the existing wastewater processing facility works based on the mains serving as the electric power source. In addition, the gaseous biomass power plant of the invention can use the organic material sources, such as food wastewater, livestock farming wastewater, agriculture waste, kitchen waste or the like, to generate a biomass gas, and thus has the dual effects of waste recovery and clean energy production.

To solve the above-identified object, the invention provides a hybrid renewable energy supply system comprising at least one renewable energy device for transforming nature energy into electric energy; and an electric energy control device, which connects the renewable energy device to a mains grid and controls storage and output of the electric energy of the renewable energy device.

In the hybrid renewable energy supply system, the at least one renewable energy device is a green power comprising a gaseous biomass energy power plant, and at least one of a wind power plant, a solar power plant or a micro-hydro power plant.

In order to process the wastewater and organic waste, the gaseous biomass energy power plant of the invention comprises: an anaerobic fermentation reactor using an organic material source to perform an anaerobic fermentation reaction to generate a biomass gas; a gas purification device performing separation, purification and collection processes on the biomass gas generated by the anaerobic fermentation reaction; a gas power plant for performing combustion-power-generation on a gas generated by the gas purification device; and a heat recovery device for recovering waste heat, generated by the combustion-power-generation, to the biomass gas generating device.

In order to maintain the working temperature of the anaerobic fermentation reactor, the anaerobic fermentation reactor of the invention further comprises a solar heater for generating heat for the anaerobic fermentation reaction.

In order to effectively process the organic wastewater and the organic waste, the anaerobic fermentation reactor of the invention comprises: a saccharification reactor comprising a stirring unit for mixing the organic material source; and a fermentation reactor for providing the anaerobic fermentation reaction to generate the biomass gas (hydrogen or methane) and a waste liquid. The material source used by the saccharification reactor is an organic substance.

In order to provide the better anaerobic fermentation reaction, the anaerobic fermentation reactor of the invention further comprises: a nutrient reactor comprising a stirring unit for providing the organic material source and a nutrient; and a mixing tank comprising a stirring unit for mixing the organic material source with the nutrient.

In this the invention, the fermentation reactor utilizes the anaerobe to perform the anaerobic fermentation reaction to generate the hydrogen or methane, and may be any conventional fermentation reactor including, for example but without limitation to, a continuous-stirred tank reactor (CSTR), an anaerobic sequencing batch reactor (ASBR), an anaerobic baffled bioreactor (ABR), a carrier-induced granular sludge bed (CIGSB), an upflow anaerobic sludge blanket (UASB), an expended granular sludge bed (EGSB), or an agitated granular sludge bed (AGSB).

In order to provide the preferred saccharification reaction, the organic material source of the invention in the saccharification reactor is an artificial substratum or an organic waste material. The artificial substratum comprises glucose, xylose, sucrose, maltose, amylase or a combination thereof. The organic waste material is organic wastewater and organic waste. The organic waste comprises wastewater sludge of a sewage treatment plant, wastewater sludge of fermentation industry, wastewater sludge of sugar industry, wastewater sludge of a food factory or the like.

In order to provide the better purification gas, the gas purification device of the invention comprises: a gas-liquid separator for separating the biomass gas from the waste liquid; and a gas purification and collection processor for purifying, collecting and processing the gases separated by the gas-liquid separator.

In the above-mentioned renewable energy supply systems, the gas power plant may also be a fuel cell power plant in addition to the combustion-power-generation.

Further aspects, objects, and desirable features of the invention will be better understood from the detailed description and drawings that follow in which various embodiments of the disclosed invention are illustrated by way of examples.

### BRIEF DESCRIPTION OF THE DRAWINGS

FIG. 1 is a schematic illustration showing a hybrid renewable energy supply system of the invention.

FIG. 2 is a schematic illustration showing an anaerobic fermentation reactor and a gas purification device of the invention.

### DETAILED DESCRIPTION OF THE PREFERRED EMBODIMENTS

The invention provides a hybrid renewable energy supply system, which may be applied to a stationary or mobile actual mass-production power generating operation, transforms a lot of organic waste into electric energy, and may be combined with green power renewable energy, such as solar energy, wind energy, micro-hydro energy or the like. The hybrid renewable energy supply system can provide the effects of waste recovery and clean energy production.

The contents and characteristics of the invention will be further described with reference to one preferred embodiment.

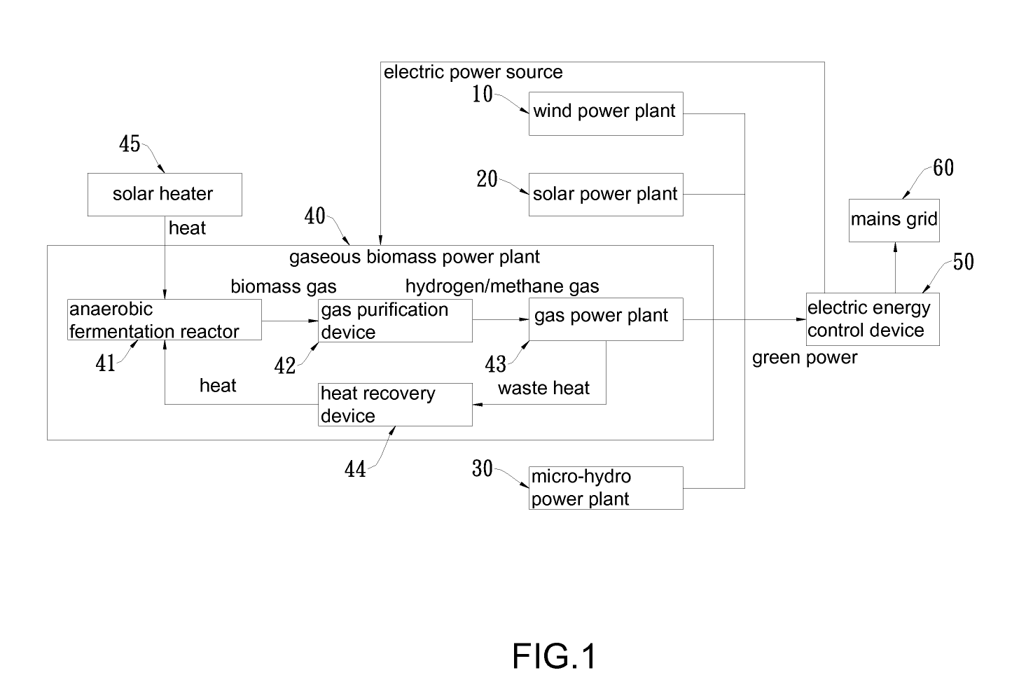

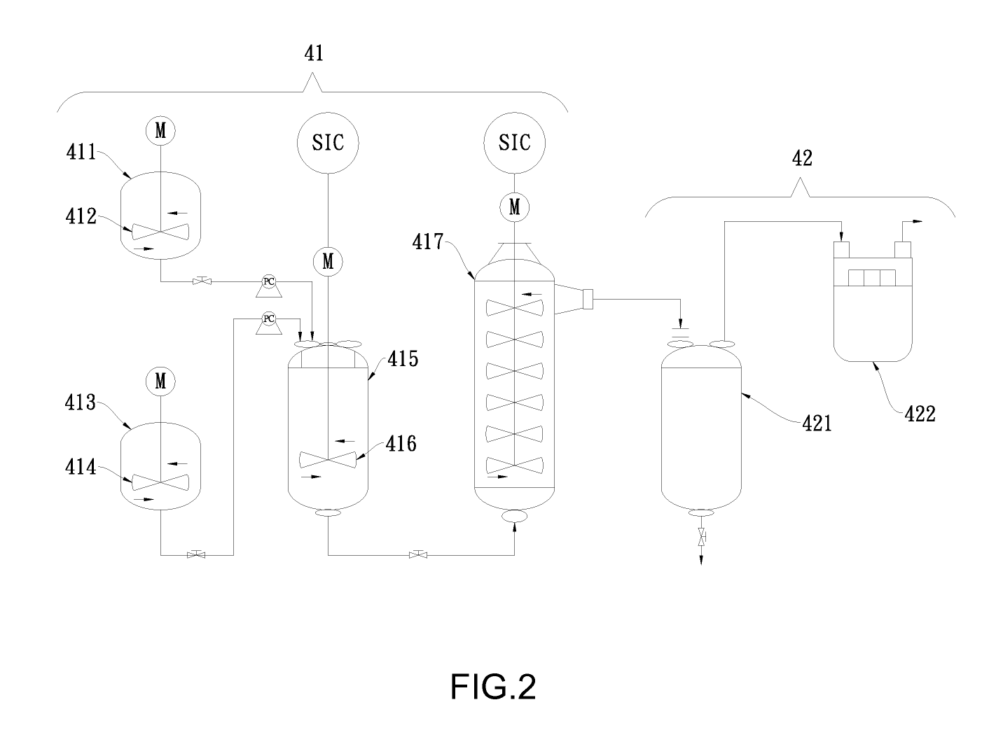

FIG. 1 is a schematic illustration showing a hybrid renewable energy supply system of the invention. FIG. 2 is a schematic illustration showing an anaerobic fermentation reactor and a gas purification device of the invention.

In FIG. 1, the hybrid renewable energy supply system of the invention includes a wind power plant 10, a solar power plant 20, a micro-hydro power plant 30, a gaseous biomass energy power plant 40, an electric energy control device 50 and a mains grid 60. The gaseous biomass energy power plant 40 comprises an anaerobic fermentation reactor 41, a gas purification device 42, a gas power plant 43, a heat recovery device 44 and a solar heater 45.

As shown in FIG. 1, the green power comprises the electric energy, which is generated by the renewable energy devices, such as the wind power plant 10, the solar power plant 20, the micro-hydro power plant 30, the gaseous biomass energy power plant 40 and the like, and is evaluated and fed back to the mains grid 60 from the electric energy control device 50. The electric power required by the gaseous biomass energy power plant 40 is provided by the wind power plant 10, the solar power plant 20, the micro-hydro power plant 30 or the power plant 40 itself, and can perform self-operating without the mains generated by the fossil energy. The combustion waste heat of the gas power plant 43 is recovered by the heat recovery device 44 and then provided to the anaerobic fermentation reactor 41. The heat required by the anaerobic fermentation reactor 41 may also be provided by the solar heater 45 without using the electric power provided by the mains grid 60.

FIG. 2 shows the anaerobic fermentation reactor 41 of the invention comprising a stirring unit 412 disposed inside a saccharification reactor 411, a stirring unit 414 disposed inside a nutrient reactor 413, a stirring unit 416 disposed inside a mixing tank 415, and a fermentation reactor 417.

In FIG. 2, the saccharification reactor 411 and the nutrient reactor 413 are connected to the mixing tank 415 and can inject the organic material source and the nutrient into the mixing tank 415, in which the organic material source and the nutrient are mixed. The mixing tank 415 is connected to the fermentation reactor 417, which can generate a biomass gas by the anaerobic fermentation reaction.

In FIG. 2, the gas purification device 42 comprises a gas-liquid separator 421 and a gas purification and collection processor 422. The gas-liquid separator 421 can separate the biomass gas, generated by the anaerobic fermentation reaction, into a biomass gas and a waste liquid. The waste liquid may drain from the gas-liquid separator 421. The biomass gas is purified, collected and processed into hydrogen or methane gas by the gas purification and collection processor 422. The hydrogen or methane gas, generated by the gas purification device 42, is again provided to the gas power plant 43 for combustion-power-generation, as shown in FIG. 1. In addition, the gas power plant 43 may also use a hydrogen fuel cell as a fuel cell power plant.

The fermentation reactor 417 in FIG. 2 mainly utilizes the anaerobe to perform the anaerobic fermentation reaction to generate the hydrogen or methane biomass gas. The fermentation reactor 417 may be a continuous-stirred tank reactor (CSTR), an anaerobic sequencing batch reactor (ASBR), an anaerobic baffled bioreactor (ABR), a carrier-induced granular sludge bed (CIGSB), an upflow anaerobic sludge blanket (UASB), an expended granular sludge bed (EGSB) or an agitated granular sludge bed (AGSB).

In FIG. 2, the organic material source in the saccharification reactor 411 is an artificial substratum or an organic waste material. The artificial substratum is glucose, xylose, sucrose, maltose, amylase or a combination thereof. The organic waste material is organic wastewater and organic waste. The organic waste comprises kitchen waste, wastewater sludge of a sewage treatment plant, wastewater sludge of fermentation industry, wastewater sludge of sugar industry, wastewater sludge of a food factory or other wastewater sludge.

New characteristics and advantages of the invention covered by this document have been set forth in the foregoing description. It is to be expressly understood, however, that the drawings are for the purpose of illustration only and are not intended as a definition of the limits of the invention. Changes in methods, shapes, structures or devices may be made in details without exceeding the scope of the invention by those who are skilled in the art. The scope of the invention is, of course, defined in the language in which the appended claims are expressed.

What is claimed is:

1. A hybrid renewable energy supply system, comprising:
at least one renewable energy device for transforming nature energy into electric energy; and
an electric energy control device, which connects the renewable energy device to a mains grid, and controls storage and output of the electric energy of the renewable energy device.

2. The hybrid renewable energy supply system according to claim 1, wherein the at least one renewable energy device is a green power comprising a gaseous biomass energy power plant and at least one of a wind power plant, a solar power plant and a micro-hydro power plant.

3. The hybrid renewable energy supply system according to claim 2, wherein the gaseous biomass energy power plant comprises:
an anaerobic fermentation reactor generating a biomass gas using an organic material source to perform an anaerobic fermentation reaction;
a gas purification device for performing separation, purification and collection processes on the biomass gas generated by the anaerobic fermentation reaction;
a gas power plant for performing combustion-power-generation on a gas generated by the gas purification device; and
a heat recovery device for recovering waste heat, generated by the combustion-power-generation, to the anaerobic fermentation reactor.

4. The hybrid renewable energy supply system according to claim 3, wherein the gaseous biomass energy power plant further comprises a solar heater for providing heat to the anaerobic fermentation reactor.

5. The hybrid renewable energy supply system according to claim 3, wherein the anaerobic fermentation reactor comprises: a saccharification reactor comprising a stirring unit for mixing the organic material source; a fermentation reactor for providing the anaerobic fermentation reaction to generate the biomass gas and a waste liquid.

6. The hybrid renewable energy supply system according to claim 3, wherein the anaerobic fermentation reactor further comprises: a nutrient reactor, which comprises a stirring unit for providing the organic material source and a nutrient; and a mixing tank comprising a stirring unit for mixing the organic material source with the nutrient.

7. The hybrid renewable energy supply system according to claim 5, wherein the fermentation reactor is a continuous-stirred tank reactor (CSTR), an anaerobic sequencing batch reactor (ASBR), an anaerobic baffled bioreactor (ABR), a carrier-induced granular sludge bed (CIGSB), an upflow anaerobic sludge blanket (UASB), an expended granular sludge bed or an agitated granular sludge bed (AGSB).

8. The hybrid renewable energy supply system according to claim 5, wherein the organic material source in the saccharification reactor is one of an artificial substratum and an organic waste material.

9. The hybrid renewable energy supply system according to claim 8, wherein the artificial substratum comprises glucose, xylose, sucrose, maltose, amylase or a combination thereof.

10. The hybrid renewable energy supply system according to claim 8, wherein the organic waste material is organic wastewater and organic waste.

11. The hybrid renewable energy supply system according to claim 10, wherein the organic waste is one of wastewater sludge of a sewage treatment plant, wastewater sludge of fermentation industry, wastewater sludge of sugar industry and wastewater sludge of a food factory.

12. The hybrid renewable energy supply system according to claim 3, wherein the gas purification device comprises:
a gas-liquid separator for separating the biomass gas from a waste liquid; and
a gas purification and collection processor for purifying, collecting and processing gases separated by the gas-liquid separator.

13. The hybrid renewable energy supply system according to claim 3, wherein the gas power plant is a fuel cell power plant.
